SEQUENCE LISTING (5 sequences)

SEQ ID NO: 1 - DNA, 387 bp, Bacillus amyloliquefaciens
aaggtagccgtatcggaaggtgcggctggatcacctcctttctaaggattttaacggaatataagaccttgggtcttataaacagaacgttccctgtcttgtttagttttgaaggatcattccttcaaaacataggaacacctgctaaatgcgccacttcctgtggcaacgcatgtgttagcatgtcctatgcgttgttctttgaaaactagataacagaagtaattcacattcaattagtaatgcaagatatcacgtagtgattctttttaacggttaagttagaaagggcgcacggtggatgccttggcactaggagccgatgaaggacgggacgaacaccgatatgcttcggggagctgtaagcaagctttgatccggagattt

SEQ ID NO: 2 - DNA, 329 bp, Bacillus amyloliquefaciens
aaggtagccgtatcggaaggtgcggctggatcacctcctttctaaggattttaacggaatataagaccttgggtcttataaacagaacgttccctgtcttgtttagttttgaaggatcattcgattcttcgagatgttgttctttgaaaactagataacagaagtaattcacattcaattagtaatgcaagatatcacgtagtgattctttttaacggttaagttagaaagggcgcacggtggatgccttggcactaggagccgatgaaggacgggacgaacaccgatatgcttcggggagctgtaagcaagctttgatccggagattt

SEQ ID NO: 3 - DNA, 1508 bp, Bacillus velezensis
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggta

SEQ ID NO: 4 - DNA, 928 bp, Bacillus amyloliquefaciens
gcgttatcgtatcccgggcgcttccggatgtgcgtgacggtctgaagccggttcacagacggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagacacgattgactatcaagataactatgacggttcagaaagagagcctgccgtcatgccttcgagatttccgaatctgctcgtaaacggggctgccggtattgcggtcggaatggcgacaaacattcccccgcatcagcttggggaagtcattgaaggcgtgcttgccgtaagtgagaatcctgagattacaaaccaggagctgatggaatacatcccgggcccggattttccgactgcaggtcagattttgggccggagcggcatccgcaaggcatatgaatccggacggggatcaatcacgatccgggctaaggctgaaatcgaagagacttcatcgggaaaagaaagaattattgtcacggaacttccttatcaggtgaacaaagcgagattaattgaaaaaatcgcggatcttgtccgggacaaaaaaatcgaaggaattaccgatctgcgagacgaatccgaccgtaacggaatgagaatcgtcattgagatccgccgtgacgccaatgctcacgtcattttgaataacctgtacaaacaaacggccctgcagacgtctttcggaatcaacctgctggcgct

SEQ ID NO: 5 - DNA, 928 bp, Bacillus velezensis
gcgttatcgtttcccgggcgcttccggatgtgcgtgacggtctgaagccggttcacagacggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagacacgattgactatcaagataactatgacggttcagaaagagagcctgccgtcatgccttcgagatttccgaatctgctcgtaaacggggctgccggtattgcggtcggaatggcgacaaacattcccccgcatcagcttggggaagtcattgaaggcgtgcttgccgtaagtgagaatcctgagattacaaaccaggagctgatggagtacatcccgggcccggattttccgactgcaggtcagattttgggccggagcggcatccgcaaggcatatgaatccggacggggatcaatcacaatccgggctaaggctgaaatcgaagagacttcatcgggaaaagaaagaattattgtcacggaacttccttatcaggtgaacaaagcgagattaattgaaaaaatcgcggatcttgtccgggacaaaaaaatcgaaggaattaccgatctgcgagacgaatccgaccgtaacggaatgagaattgtcattgagatccgccgtgacgccaatgctcacgtcattttgaataacctgtacaaacaaacggccctgcagacgtctttcggaatcaacctgctggcgct

(19) United States
(12) Patent Application Publication
(10) Pub. No.: US 2021/0171877 A1
(43) Pub. Date: Jun. 10, 2021

(54) Detergent Composition

(71) Applicant: ETS POLLET S.A., Tournai (BE)

(72) Inventor: Abdelhamid Jabrane, Beersel (BE)

(21) Appl. No.: 16/768,732

(22) PCT Filed: Dec. 7, 2018
(86) PCT No.: PCT/EP2018/084001

§ 371 Date: Jun. 1, 2020

(30) Foreign Application Priority Data
Dec. 8, 2017 (BE) 2017/5916
Jul. 12, 2018 (BE) 2018/55014

(51) Int. Cl. C11D 3/38; C12Q 1/18

(52) U.S. Cl. CPC C11D 3/381; C12Q 1/18

(57) ABSTRACT

A liquid detergent composition comprising bacteria able to interact with the growth of pathogenic and/or of undesirable microorganisms and use of this composition.

### TECHNICAL FIELD OF THE INVENTION

The present invention relates to detergent compositions having the property of specifically inhibiting the growth of pathogens or undesirable.

### TECHNOLOGICAL BACKGROUND OF THE INVENTION

Pathogenic bacteria are a health hazard. This danger, due to their pathogenic power, is amplified by their multi resistance to antibiotics. Indeed, the use, even overuse, of antibiotics has led to the emergence of multi-resistant pathogenic strains.

There is therefore a need for compositions capable of limiting the emergence of bacteria resistant to antibiotics. Disinfectants can present a solution but with toxicological and ecotoxicological risks that these disinfectants present.

The patent application WO 2014/079938 describes the addition of enzymes to detergent compositions for the treatment of biofilms resistant to conventional disinfectants and/or biocides.

The patent application US 2008/0233093 describes the use of very specific strains of Bacillus ssp to reduce the formation of biofilms.

US 2010/0028314 describes the use of the NRRL B-50154 strain of Bacillus amyloliquefaciens in the treatment of biofilms and its use according to several distinct embodiments, which are linked to the secretion of amylase by this bacterial strain.

Similarly, US 2010/0008893 describes the use of the NRRL B-50150 strain of Bacillus velezensis in the treatment of biofilms, here also thanks to the secretion of amylase by this bacterial strain.

Finally, US 2011/0230345 describes the use of the NRRL B-50349 strain of Bacillus amyloliquefaciens for phytosanitary use.

No prior art document describes the addition of bacteria to detergent compositions further comprising a preservative for the purpose of specifically interacting with pathogens.

### BRIEF DESCRIPTION OF THE INVENTION

The present invention relates to a method for the identification of bacteria capable of interacting with the growth of pathogenic or undesirable microorganisms, comprising the following successive steps:
obtaining a bacteria to be tested;
obtaining one or more pathogenic or undesirable microorganism(s);
determining the culture conditions allowing the growth of both said pathogenic or undesirable microorganism(s) and said bacteria (to be tested) and/or obtaining a culture supernatant of said bacteria (to be tested);
determining the growth of said pathogenic or undesirable microorganism(s) (i) in the absence of said bacteria (to be tested) and (ii) in the presence of said bacteria (to be tested) or of said culture supernatant of said bacteria (to be tested);
identifying bacteria which causes a specific reduction in the growth of at least one of said pathogenic or undesirable microorganism(s).

Preferably, in the method, the pathogen(s) is (are) selected from Gram-bacteria, Gram+ bacteria, molds and yeasts.

Advantageously, at least 2, 3, 4 or 5 pathogenic or undesirable microorganisms are selected.

Preferably, the pathogenic or undesirable microorganism(s) include Escherichia coli, Staphyloccocus aureus (ex. MRSA), and another pathogen selected from Pseudomonas aeruginosa, a yeast and a fungus, preferably where one, 2, or each of said pathogens is (are) resistant to antibiotic treatment. The growth of other pathogenic microorganisms, such as Salmonella enterica (subs. Enterica) and Streptococcus pyogenes is also reduced by the composition of the present invention.

Advantageously, pathogens are known to cause diseases, for example nosocomial diseases. Preferably, the bacteria (to be tested) is from the Bacillus amyloliquefaciens group.

Preferably at least 100 contiguous nucleotides of the genome of the bacteria (to be tested) has at least 75% identity with at least 100 contiguous nucleotides of SEQ ID NO:1, SEQ ID NO:3, SEQ ID NO:4 and/or SEQ ID NO:5.

A related aspect of the invention relates to the use of the bacteria obtainable by the above method (in particular a bacteria from the group Bacillus amyloliquefaciens and/or a bacteria having at least 75% identity with at least 100 contiguous nucleotides of SEQ ID NO:1, SEQ ID NO:3, SEQ ID NO:4 and/or of SEQ ID NO:5), in a detergent composition, preferably for use in cleaning surfaces and/or floors and/or contact points.

Such use is advantageous for communities, such as hospitals, nurseries for young children, nursing homes for the elderly or revalidation, department stores, canteens and kitchens.

Another related aspect of the present invention is the use of a liquid detergent composition of pH between 2.5 and 11.5 comprising (i) bacteria, (ii) one or more molecule(s) having surface properties and/or surfactant and/or amphiphilic and (iii) a preservative for the reduction of pathogenic and/or nosocomial bacteria present on a surface and/or contact points.

For this use, preferably, the bacteria is obtainable by the method described above (in particular bacteria of the Bacillus amyloliquefaciens group and/or those having at least 75% identity with at least 100 contiguous nucleotides of SEQ ID NO:1, SEQ ID NO:3, SEQ ID NO:4 and/or SEQ ID NO:5).

For this use, preferably, the preservative is chosen from the group consisting of isothiazolinone, formaldehyde and formaldehyde-generating molecules, preservatives with an amine function, preservatives with an alcohol function, benzoate and its derivatives, parabens and derivatives, methyldibromoglutaronitrile, sodium hydroxy methyl glycinate, triclosan and Sorbate (and/or sorbic acid).

Another related aspect of the present invention is a liquid detergent composition comprising one or more molecule(s) having surface and/or surfactant and/or amphiphilic properties of between 1% and 30% by weight quantity; one or more preservative(s) of between 0.001% and 10% by weight quantity and bacteria obtainable by the method described above (in particular a bacterium of the Bacillus amyloliquefaciens group and/or a bacteria with at least 75% identity with at least 100 contiguous nucleotides of SEQ ID NO:1, SEQ ID NO:3, SEQ ID NO:4 and/or SEQ ID NO:5).

Preferably, the preservative of the detergent composition is chosen within the group consisting of isothiazolinone, formaldehyde and formaldehyde-generating molecules, preservatives with an amine function, preservatives with an alcohol function, benzoate and its derivatives, parabens and derivatives, quaternary ammoniums, methyldibromoglutaronitrile, Sodium hydroxy methyl glycinate, triclosan and Sorbate (and/or sorbic acid).

Preferably, in the detergent composition, the bacteria are present at a concentration of at least 200,000 cfu/ml, preferably at least 1,000,000 cfu/ml, even more preferably at a concentration of at least 10,000,000 cfu/ml.

Advantageously, the detergent composition further comprises one or more compound(s) chosen from (mono-) nucleotides (or nucleosides), a phytohormone, mineral nitrogen and a carbon source.

Another related aspect of the present invention is a liquid composition comprising at least 200,000 cfu/ml, preferably at least 1,000,000 cfu/ml, even more preferably at least 10,000,000 cfu/ml of bacteria having at least 100 contiguous nucleotides of its genome having at least 75% of identity with at least 100 contiguous nucleotides of SEQ ID NO:1 or SEQ ID NO:3 and/or SEQ ID NO:4 and/or SEQ ID NO:5.

Advantageously, this liquid composition also comprises a preservative (chosen from the group consisting of isothiazolinone, formaldehyde and formaldehyde-generating molecules, preservatives with an amine function, preservatives with an alcohol function, benzoate and its derivatives, parabens and derivatives, quaternary ammoniums, methyldibromoglutaronitrile, Sodium hydroxy methyl glycinate, triclosan and Sorbate (and/or sorbic acid)) and/or one or more compound (s) chosen from nucleotides, a phytohormone, mineral nitrogen and a carbon source.

### BRIEF DESCRIPTION OF THE FIGURES

FIG. 1 describes the interaction between the bacteria of the invention (BS) and Candida albicans (CA) in liquid culture.

FIG. 2 describes the interaction between the bacteria of the invention (BS) and Staphylococcus aureus (methicillin resistant strain; MRSA) in liquid culture.

FIG. 3 describes the interaction between the bacteria of the invention (BS) and Escherichia coli (EC) in liquid culture.

### DETAILED DESCRIPTION OF THE INVENTION

The inventor has noticed, surprisingly, that the enrichment of specific bacteria to a detergent composition intended to be applied to a surface reduces the amount of pathogens on this surface without there any overall biocidal or disinfectant effect.

In addition, the inventor has been successful in introducing such bacteria into detergent compositions, both in their concentrated and diluted forms, being compatible with the metabolism of these bacteria.

A first aspect of the invention is therefore a method for identifying bacteria capable of interacting with the growth of pathogens (or harmful microorganisms) comprising the following successive steps:

a bacteria to be tested (identified) or a collection of bacteria to be tested (identified) is obtained (selected);

one or more pathogens (and/or harmful microorganisms) are obtained (selected);

the culture conditions allowing the growth of both this pathogen(s) (and/or harmful microorganism) and this bacteria are determined and/or a culture supernatant of this bacteria to be identified is taken;

the growth of the pathogen (s) (and/or harmful microorganism) (i) in the absence of this bacteria and (ii) in the presence of this bacteria is determined and/or the growth of the pathogen in the presence culture supernatant of this bacteria is determined;

a bacteria is identified, which causes a specific reduction in the growth of at least one of the pathogens.

In the context of the present invention, the terminology “(specific) reduction of growth” means that the quantity of the pathogen in condition (ii), therefore in the presence of the bacteria to be tested or of the culture supernatant of the bacteria to be tested, is lower than in condition (i), therefore in the absence of the bacteria to be tested. Advantageously, the quantity of the pathogen after culture under condition (ii) can also be lower than the quantity seeded.

In the context of the present invention, the terminology “specific reduction (of growth)” means that the bacteria do not have a general biocidal effect. The bacteria of the invention can, for example, act by competition with the pathogen (or the harmful microorganism), or produce a factor which specifically disturbs the metabolism of the pathogen (or the harmful microorganism).

Measurement (ii) above can be done in several ways which can be cumulated. In particular, it is possible to obtain a supernatant from a culture of the bacteria to be tested and to measure the effect of this supernatant on the growth of pathogens or of the harmful microorganism (in monoculture).

An alternative is to carry out a co-culture test where a predetermined quantity of the bacteria and of the pathogen (or the harmful microorganism) are seeded.

Advantageously, different quantity ratios between the bacteria and the pathogen are tested. The pathogen is advantageously grown on a surface (but preferably not as a biofilm). This makes it possible to highlight a possible phenomenon of competition for the substrate, or even the presence of an inhibiting factor which would be produced by the bacteria to be tested (e.g. presence of an inhibition halo).

However, a co-culture test in liquid medium is possible. Advantageously, the two tests, on surface and in liquid medium, are performed.

In the case of coculture in a liquid medium, it is advantageous to measure the optimal amount of bacteria to be seeded, or the optimal ratio (amount of bacteria to be seeded:amount of pathogen) or, at a minimum, to test 3, 4, 5 or even 10 different ratios, presenting a range of variation by a factor of at least 10 (for example 1:1 and 20:1).

A preferred culture medium for co-culture comprises yeast extracts, protein hydrolysates (peptone) and metabolizable sugars, for example glucose. Preferably, the pH of the medium is controlled and maintained during the culture. Those skilled in the art know how to determine the optimum pH, both for simple culture and for co-culture.

In the context of the present invention, the bacteria to be tested can be any bacteria, which would not penalize the properties of the detergent composition. Thus, these bacteria have no known toxicity to humans or animals and have no general biocidal effect. These bacteria are preferably natural, that is to say that they are not modified by genetic engineering.

A preferred strain of these bacteria is found in the Bacillus subtilis complex. Preferably Bacillus amyloliquefaciens or Bacillus velezensis. More preferably, the bacteria to be tested have an element of their genome having at least 70%, 75%, 80%, 85%, 90%, 91%, 92%, 93%, 94%, (approximately) 95%, 96%, 97%, 98%, 99% or even have 100% identity with at least 100 contiguous nucleotides of the ribosomal RNA 16S or 23S of Bacillus amyloliquefaciens (e.g. strains ATCC 39374 and 39320; see SEQ ID NOs: 1, 2) or from Bacillus velezensis (e.g. strain NRRL B-41580; SEQ ID NO: 3), or having at least 70%, 75%, 80%, 85%, 90%, 91%, 92%, 93%, 94%, (approximately) 95%, 96%, 97%, 98%, 99% or even have 100% identity with at least 100 contiguous nucleotides of the DNA of the in the Gyrase alpha unit (see SEQ ID NO: 4, Bacillus amyloliquefaciens strain CP 1604 and/or SEQ ID NO: 5, Bacillus velezensis, strain B-41580). Such a sequence comparison can advantageously be done using BLASTn, well known to those skilled in the art.

A related aspect of the present invention is that of the use of the bacteria described above to reduce, for example on a surface, the quantity of nosocomial bacteria, yeasts and/or fungi and/or that can cause diseases and/or are unwanted.

These are advantageously chosen from the group consisting of Actinomyces, Bacillus anthracis, Brucella, Campylobacter, Clostridium sp. (C. botuminium, C. perfringens, C. tetani), Borrelia sp., Corynebacterium sp., Enterobacter sp., Escherichia coli, Flavobacterium meningosepticum, Haemophilus sp., Helicobacter pylori, Klebsiella sp., Legionella sp., Listeria ivanovii, Listeria monocytogenes, Mycobacterium sp., Neisseria meningitidis, Pasteurella sp., Proteus mirabilis, Pseudomonas aeruginosa, Salmonella sp. (ex. S. typhimurium), Shigella dysenteriae, Staphylococcus aureus, Streptococcus sp. (ex. S. pneumoniae or S. pyogenes), Vibrio sp. (ex. V. cholerae), Yersinia sp. (ex. Y. pestis and Y. enterolitica), Aspergillus fumigatus, Candida albicans, Trichphyton sp., Bacillus cereus, Cladosporidium sp., Penicillium sp., Aspergillus sp., Alternaria alternata and Ulocladium botrytis.

Among the bacteria, yeasts and/or fungi which are nosocomial and/or which can cause diseases, there are in particular certain strains of Escherichia coli, of Staphyloccocus aureus, in particular the strains resistant to methicillin (MRSA), of Pseudomonas aeruginosa and Candida albicans, or even Salmonella enterica (subs. enterica) and Streptococcus pyogenes.

Another related aspect of the present invention is the use (or incorporation) of the bacteria identified above in detergent compositions.

The preferred detergent compositions are those for the treatment of surfaces, in particular the surfaces of communities such as hospitals, nurseries for young children, nursing homes for the elderly or in rehabilitation, department stores (including handles of shopping carts), canteens.

This advantageously makes it possible to clean these surfaces, while directly reducing the abundance of pathogens which would be present there.

In the context of the present invention, the detergent compositions are not particularly limited.

However, the detergent compositions are liquids. They preferably have the pH (in general the detergent compositions contain water, which does not exclude the presence, even the majority, of organic solvents) and the various compounds in concentration which are compatible with the life of the bacteria (described above) for a sufficiently long period (for example several weeks or more; in general, the compositions are stable for several months, for example 10 months or more).

If this is not possible, the bacteria of the invention can be formulated in an intermediate composition, preferably liquid, which must be mixed just before use with the detergent composition, preferably in simple proportions (e.g. between 1:100 and 100:1, preferably between 1:10 and 10:1).

In the context of the present invention, the detergent composition is a liquid solution (aqueous and/or comprising one or more organic solvents) and this solution comprises (in addition to the bacteria described above) at least one molecule having surface and/or surfactant and/or amphiphilic properties such as surfactants and soaps (preferably less than 30% by weight) and one (at least one) preservative (and/or stabilizer) (up to 10% by weight for the total of the preservatives present). When the detergent composition is aqueous, including a composition comprising one or more organic solvents, its pH is fixed, preferably between 2.5 and 11.5, advantageously between 4 and 10, or even between 7 and 10.

The composition also advantageously comprises waxes and/or polymers (preferably less than 30% by weight), bleaching agents (preferably less than 15% by weight), acids, bases (preferred) or salts (preferably a total of less than 30% by weight), as well as one or more compounds chosen from the group consisting of chelating agents, enzymes, thickeners and corrosion inhibitors.

The detergent composition can optionally comprise one or more perfume(s) and/or one or more coloring agent(s).

Another related aspect of the present invention is the bacteria identified above in a liquid medium at a concentration of at least 200,000 cfu/ml, for example at least 500,000 cfu/ml, at least 1 million cfu/ml, at least 5 million cfu/ml, at least 10 million cfu/ml, for example about 40 to about 70 million cfu/ml. Cfu is the acronym in English for “colony forming unit”, which is the usual way of quantifying the presence of bacteria by its capacity to generate colonies, once this bacteria has been seeded on an appropriate culture support.

Preferably, this liquid medium also comprises at least one molecule having surface and/or surfactant and/or amphiphilic properties such as surfactants and soaps (preferably less than 30% by weight) and a preservative (stabilizer) (up to 10% by weight, preferably up to 1% by weight).

The liquid medium advantageously further comprises one or more compounds for promoting the growth of the bacteria of the invention (once placed on a surface to be cleaned), preferably chosen from nucleotides (e.g.; adenine, guanine; 0.1 to 1% by weight:weight_{liquid medium}), a phytohormone, for example 3-acetic indole (from 0.01% to 0.1% in wt_{liquid medium}) mineral nitrogen (ex. NH₄SO₄: 0.1 to 1% by weight_{liquid medium}) and a carbon source (e.g. glucose). This compound for promoting the growth of the bacteria of the invention is particularly useful for formulations where the concentration of the bacteria of the invention quite low (e.g. 200,000 cfu/ml, 500,000 cfu/ml, 1,000,000 cfu/ml).

The liquid medium can be aqueous, which means that it includes water and can also include non-aqueous solvents.

A preferred liquid medium comprises at least 50% by weight (weight organic solvent:weight of all the solvents) of a solvent (that is to say a non-aqueous substance liquid at room temperature) chosen from the group consisting of alcohols (e.g. benzylic, ethanol, isopropanol), glycol ethers (e.g. butyldiglycol, monopropylene glycol, dipropylene glycol), petroleum distillates (e.g. “white spirit”, petroleum ether, paraffin oil), amino compounds (e.g. monoethanolamine), ketone and aldehyde derivatives (e.g. methylal, dimethoxy methane).

The preservative (s) (stabilizer (s)) are present at concentrations of 10% by weight maximum, preferably between 0.001% and 10%, more preferably, between 0.01% and 1%, even more preferably, between 0.05% and 0.2% (weight of the preservative (s) present (s):weight of the composition).

The preservative (s) (stabilizers) are preferably chosen from the group consisting of isothiazolinone (ex. 1,2-Benzisothiazol-3-one; 2-Methyl-2H-isothiazol-3-one; 5-Chloro-2-methyl-isothiazolin-3 (2H)-one (CMI); 2-methylisothiazolin-3 (2H)-one (MI)), formaldehyde and compounds generating formaldehyde (e.g. glutaraldehyde; 2-bromo-2-nitropropane-1,3-diol; 5-bromo-5-nitro-1,3-dioxane), preservatives (stabilizers) with an amine function (e.g. Diazolinidylurea; Chloroacetamide), preservatives (stabilizers) with an alcohol function (e.g.: Phenoxypropanol; Benzyl alcohol; o-Phenylphenol; Phenoxy-ethanol), benzoate and its derivatives, parabens and derivatives, methyldibromoglutaronitrile, sodium hydroxy methyl glycinate, triclosan and sorbate (and/or sorbic acid), as well as mixtures of several of these preservatives (stabilizers). For example a CMI+MI mixture in mass proportions of 3:1.

### EXAMPLES

### Example 1. Pathogenic Strains (SP)

| | |
|---|---|
| Candida albicans (CA) | ATCC 64124 |
| Staphyloccocus aureus (MRSA) | ATCC 33591 |
| Escherichia coli (EC) | ATCC BAA-2452 |
| Klebsiella pneumoniae (KP) | ATCC BAA-2146 |
| Pseudomonas aeruginosa (PA) | ATCC BAA-2108 |

### Example 2. Development of a Liquid Medium Common to the Bacteria of the Invention and to MS

The inventor has noticed that all of the above SP strains (even CA, the culture of which is recommended rather at pH 5.6), but also the bacteria of the invention, are capable of growing in medium A comprising yeast extract, peptone and glucose at pH 7 and at room temperature.

### Example 3. Analysis of the Interaction Between the Bacteria of the Invention and SP in Liquid Culture

The starting inoculum of the strain of the bacteria of the invention was fixed at a high concentration. To do this, a colony of the bacteria of the invention is taken from an agar dish and inoculated in 10 ml of medium A in a flask. The number of CFUs/ml is determined after an incubation of 24 h on the particle counter.

The concentration of the starting inoculum of the SP strains is 5·10² CFU/ml. Given the difficulty of measuring such low concentrations on the particle counter, several colonies are taken from an agar dish and suspended in a precise volume of medium A. This culture is then counted on the particle counter and then diluted to the desired concentration.

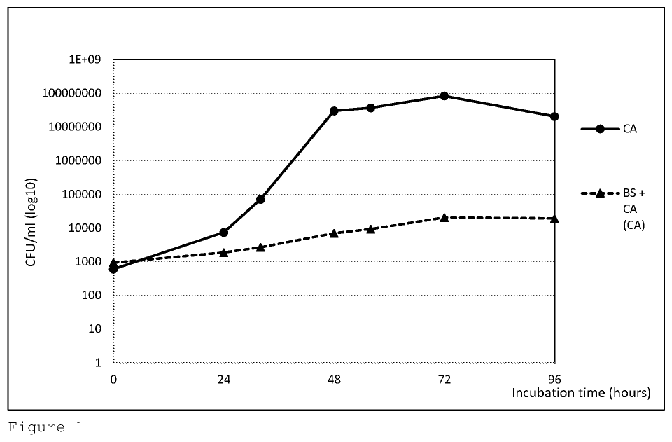

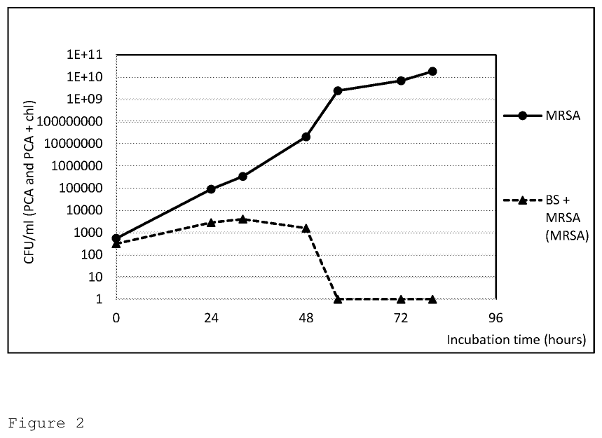

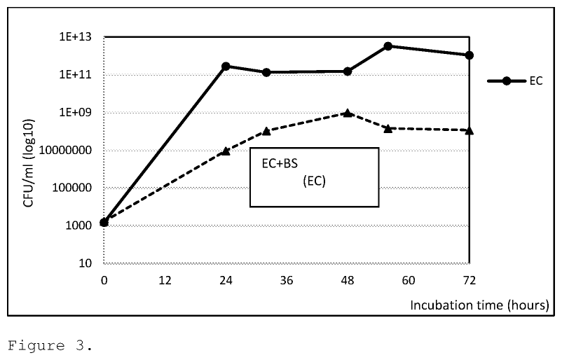

As shown in FIGS. 1-3, in liquid culture, the bacteria of the invention reduced the growth of EC, greatly reduced the growth of CA and had an even more marked effect on MRSA.

The bacteria of the invention also greatly reduced the growth in liquid medium Salmonella enterica (subs. enterica) and Streptococcus pyogenes.

On the other hand, the bacteria of the present invention had no significant effect in liquid culture on the growth of KP or of PA, which shows that the bacteria of the invention has no general biocidal properties, and suggests a specific effect.

### Example 4. Analysis of the Interaction Between the Bacteria of the Invention and SP in Solid Medium

The interaction between the bacteria of the invention and each strain of SP was studied in agar medium: on the one hand, the effect of the culture supernatant was tested on a agar culture of each SP; on the other hand, the effect of the bacteria of the invention on the growth of the SP strains was tested by spreading and streaking or by adding colonies of the bacteria of the invention to an SP monolayer.

CA and EC were grown, and show growth in monolayer. The addition of the culture supernatant of the bacteria of the invention inhibited the growth of CA and EC. An inhibitory effect was less evident in the case of the other SPs.

The measurement of interactions by streaking showed a significant growth of the bacteria of the invention, and a low number of CA colonies. The effect is even more spectacular in the case of MRSA, and also observable in the case of EC.

The inventor also put the SPs in culture on agar, then after drying, 3 colonies of the bacteria of the invention. In the case of CA, the bacteria of the invention had a significant growth, which covers CA. The inventor observed that the bacteria of the invention, where it was seeded, caused halos of growth inhibition of the MRSA. The effect is less marked for other SPs.

1. A liquid detergent composition comprising one or several molecule(s) having surface and/or surfactant and/or amphiphilic properties in a weight quantity comprised between 1% and 30%; one or several preservative(s) in a weight quantity between 0.001% and 10% and an antagonist bacteria of one or of several pathogenic or undesirable microorganism(s).

2. The liquid detergent composition of claim 1, wherein the antagonist bacteria of one or of several pathogenic or undesirable microorganism(s) acts through competition with the one or of several pathogenic or undesirable microorganism(s), or produces a factor that specifically disturbs the metabolism of the one or of several pathogenic or undesirable microorganism(s).

3. Liquid detergent composition comprising one or several molecule(s) having surface and/or surfactant and/or amphiphilic properties in a weight quantity comprised between 1% and 30%; one or several preservative(s) in a weight quantity between 0.001% and 10% and a bacteria obtainable by a method comprising the following successive steps:
obtaining a bacteria to be tested;
obtaining one or more pathogenic or undesirable microorganism(s);
determining the culture conditions allowing the growth of both said pathogenic or undesirable microorganism(s) and said bacteria and/or obtaining a culture supernatant of said bacteria;
determining the growth of said pathogenic or undesirable microorganism(s) (i) in the absence of said bacteria and (ii) in the presence of said bacteria or of said culture supernatant of said bacteria;
identifying bacteria which causes a specific reduction in the growth of at least one of said pathogenic or undesirable microorganism(s)

4. The liquid detergent composition of claim 1, wherein at least 100 contiguous nucleotides of the genome of the bacteria has at least 75% identity with at least 100 contiguous nucleotides of SEQ ID NO:1, SEQ ID NO:3, SEQ ID NO:4 and/or SEQ ID NO:5.

5. The liquid detergent composition of claim 1, wherein the bacteria is from the Bacillus amyloliquefaciens group.

6. The liquid detergent composition of claim 1, wherein the preservative is selected from the group consisting of isothiazolinone, formaldehyde and formaldehyde-generating molecules, preservatives with an amine function, preservatives with an alcohol function, benzoate and its derivatives, parabens and derivatives, quaternary ammoniums, methyldibromoglutaronitrile, Sodium hydroxy methyl glycinate, triclosan and Sorbate (and/or sorbic acid).

7. The liquid detergent composition of claim 1, wherein the bacteria are present at a concentration of at least 200,000 cfu/ml, preferably at least 1,000,000 cfu/ml, even more preferably at a concentration of at least 10,000,000 cfu/ml

8. The liquid detergent composition of claim 1, further comprising one or more compound(s) chosen from nucleotides, a phytohormone, mineral nitrogen and a carbon source.

9. The liquid detergent composition of claim 1, wherein the pathogenic or undesirable microorganism(s) include Escherichia coli, Staphyloccocus aureus (e.g. MRSA), and another pathogen selected from Pseudomonas aeruginosa, a yeast and a fungus, preferably where one, 2, or each of said pathogens is (are) resistant to an antibiotic treatment.

10. The liquid detergent composition of claim 1, wherein the pathogens are known to cause diseases, for instance nosocomial diseases.

11. A liquid detergent composition comprising at least 200000 cfu/ml, de preferably at least 1000000 cfu/ml, more preferably at least 10000000 cfu/ml of bacteria having at in their genome least 100 contiguous nucleotides having at least 75% of identity with at least 100 contiguous nucleotides from SEQ ID NO:1 or from SEQ ID NO:3 and/or from SEQ ID NO:4 and/or from SEQ ID NO:5.

12. The liquid detergent composition of claim 11 further comprising a preservative.

13. The liquid detergent composition of claim 11 further comprising one or more compound(s) chosen from nucleotides, a phytohormone, mineral nitrogen and a carbon source.

14. Use of the liquid detergent composition of claim 11 for the cleaning of surfaces, preferably for communities, preferably chosen from hospitals, nurseries for young children, nursing homes for the elderly or revalidation, department stores, canteens and kitchens.

15. A method for the cleaning of surfaces preferably present in communities, preferably chosen from hospitals, nurseries for young children, nursing homes for the elderly or revalidation, department stores, canteens and kitchens, comprising applying the liquid detergent composition of claim 11.
